(19) United States
(12) Patent Application Publication
(10) Pub. No.: US 2019/0151453 A1
(43) Pub. Date: May 23, 2019

(54) Drug/polymer Composite Materials And Methods Of Making The Same

(71) Applicant: Micell Technologies, Inc., Durham, NC (US)

(72) Inventors: James B. McClain, Ocracoke, NC (US); James P. DeYoung, Dallas, TX (US)

(73) Assignee: Micell Technologies, Inc., Durham, NC (US)

(21) Appl. No.: 16/259,541

(22) Filed: Jan. 28, 2019

Related U.S. Application Data
(63) Continuation of application No. 11/158,724, filed on Jun. 22, 2005.

(51) Int. Cl. A61K 47/32; A61K 9/16; A61K 9/00

(52) U.S. Cl. CPC A61K 47/32; A61K 9/1635; A61K 9/0024; A61K 9/1694

(57) ABSTRACT

A method of forming a drug/polymer composite material is carried out by combining a drug material with a polymer material under pressure in the presence of a compressed gas solvent (e.g., carbon dioxide) to form the drug/polymer composite material. Drug/polymer composite materials and shaped articles (e.g., subcutaneous drug depots) which may be produced by a process are also described, along with methods of use thereof.

### FIELD OF THE INVENTION

The present invention concerns methods of making drug/polymer composite materials, the materials so made, and shaped articles formed from such drug/polymer composite materials.

### BACKGROUND OF THE INVENTION

Drug/polymer composite materials are traditionally formed either by solvent-based processing where a solvent or combination of solvents is used to facilitate intimate mixing of the drug with polymer(s) by a combination of reducing the polymer viscosity and by dispersing/dissolving the drug into a fluid-like phase. The solvents commonly utilized include all common organic solvents, halogenated solvents and aqueous solvent compositions. However, Solvent-based processing can adversely affect the drug by reacting, bonding or binding with the chemical functionality of many drugs. In addition, removal of solvent and solvent residues from the composite material is problematic and requires extensive processing with heat, vacuum, etc. Further, these processes can be process/cost intensive, lack precise material control and can adversely affect the drug. For example: (i) Trace solvent residues are unavoidable and are often toxic or can negatively interact with the drug or polymer molecules altering the therapeutic effect (ii) Solvent-based processing can also adversely affect the primary structure of the drug in the polymer matrix. For example, making very difficult the production of small particles/domains of drug in the polymer matrix. (iii) Solvent-based processing can also adversely affect the secondary structure of sophisticated therapeutics such as proteins, enzymes, hormones, which changes the drug's efficacy and may denature the drug compound rendering it useless or toxic or change its effective shelf-life. (iii) Solvent-based processing can also adversely affect the polymorph of the drug; changing crystalline structure or providing amorphous materials that have different bioavailability profiles and adversely affecting shelf-life.

An alternative traditional process uses elevated temperatures to provide a lower viscosity polymer(s) for mixing with the drug. Again, however, high temperature processing can adversely affect many thermally sensitive drugs, rendering them ineffective or toxic, and elevated temperature processing is often used in conjunction with solvent-based methods (one still has to dissolve/disperse the drug molecule(s)), resulting in combined challenges of high temperature and solvents.

Densified gases, liquid and supercritical fluids have been described in the art as processing media for the incorporation of active materials including drugs into polymeric matrices. U.S. Pat. No. 5,340,614 (Perman) describes impregnating materials into polymeric matrices by using a carrier liquid that carries the active ingredient(s) where the carrier fluid is substantially insoluble in the supercritical fluid as is the active ingredient(s). A polymeric material is added to a pressure vessel after which the carrier liquid containing the active material(s) is(are) added, and then the system is exposed to supercritical carbon dioxide. After removal of the supercritical fluid and the carrier fluid, the polymer is found to have absorbed a portion of the active and presumably the carrier fluid.

U.S. Pat. No. 6,190,699 (Luzzi) describes compositions of protein and peptide infused polymer particles and methods for production using compressed solvents including supercritical fluids. Luzzi claims that the proteins and peptides are partially adsorbed into (infused) the polymer particles. Since proteins and peptides are not soluble in supercritical carbon dioxide, it can be reasonable assumed that dense carbon dioxide is not a suitable compressed solvent to practice this art as sorption would be disfavored due to a lack of solubility of the protein in the compressed solvent. Additionally, Luzzi discloses methods for making particles and does not address shaped or formed articles or semi-porous or porous articles.

What is needed in the art is a method that allows for the formation of polymer-drug composites that does not require the use of a carrier liquid or emulsions to make soluble or make mobile the drug for addition to the polymer. What is needed in the art is a method that allows for production of a polymer-drug composite that does not physically or chemically change the state of the drug during processing (solid to liquid). What is needed in the art is a method that allows for the creation of formed articles of a desired and controllable geometry. What is needed in the art is a method that allows for a low temperature forming of a semi-porous or porous solid article that does not physically or chemically change the state of the drug during processing.

Accordingly, there is a need for new approaches to the production of drug/polymer composite materials, and for new materials produced by such methods.

### SUMMARY OF THE INVENTION

A first aspect of the present invention is a method of forming a drug/polymer composite material by combining a drug material with a polymer material under pressure in the presence of a compressed gas solvent to form the drug/polymer composite material.

A further aspect of the present invention is a drug/polymer composite material (in some embodiments a “medicament” herein), which may be produced by a process as described above.

A further aspect of the present invention is a shaped article (in some embodiments also referred to as a “medicament” herein) comprising, consisting of or consisting essentially of a drug/polymer composite material as described above.

A further aspect of the present invention is a method of treating a subject with a drug, comprising administering a drug/polymer composite material as described herein to said subject in an amount effective to treat said subject with said drug.

A further aspect of the present invention is the use of a drug for the preparation of a medicament for carrying out a method of treatment as described herein.

The foregoing and other objects and aspects of the present invention are explained in greater detail in the drawings herein and the specification set forth below.

### DETAILED DESCRIPTION OF THE PREFERRED EMBODIMENTS

The present invention, is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

The disclosures of all United States patents cited herein are to be incorporated herein by reference in their entirety.

### A. Definitions.

Subjects that may be treated by the present invention include both human subjects for medical purposes and animal subjects for veterinary and drug screening and development purposes. Other suitable animal subjects are, in general, mammalian subjects such as primates, bovines, ovines, caprines, porcines, equines, felines, canines, rodents (e.g., rats and mice), etc. Human subjects are the most preferred. Human subjects include fetal, neonatal, infant, juvenile and adult subjects.

“Polymer” as used herein refers to organic polymers, and includes copolymers of a named polymer with other constituents. In sonic embodiments, such as in the preparation of drug depots or drug delivery devices, the polymer is preferably an absorbable and/or resorbable polymer. In other embodiments the polymer is preferably non-resorbable and biocompatible.

Shaped articles as used herein include, but are not limited to, pills, tablets, drug depots or drug delivery devices (e.g., subcutaneous implants), biomedical implants, etc.

“Biomedical implant” as used herein includes but is not limited to stents (e.g., vascular stems), electrodes, catheters, leads, implantable pacemaker or cardioverter housings, joints, screws, rods, ophthalmic implants (including, but not limited to, intraocular lens implants, glaucoma implants or drainage implants, and punctal implants or plugs), etc. The implants may be of any suitable material, including but not limited to organic polymers (including stable or inert polymers and biodegradable polymers), metals such as stainless steel and titanium, inorganic materials such as silicon, and composites thereof.

“Drug depot” or “drug delivery device” include those be configured for any route of administration, including those that may be implanted (luminal, venous, subcutaneous, muscular, ocular), inserted (oral, rectal, vaginal, ocular) or topically applied (transdermal, transmucual, sublingual).

“Treat” as used herein refers to any type of treatment or prevention that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc. As such, the term “treatment” also includes prophylactic treatment of the subject to prevent the onset of symptoms. As used herein, “treatment” and “prevention” are not necessarily meant to imply cure or complete abolition of symptoms.” to any type of treatment that imparts a benefit to a patient afflicted with a disease, including improvement in the condition of the patient (e.g., in one or more symptoms), delay in the progression of the disease, etc.

“Pharmaceutical excipient” as used herein includes refers to any pharmaceutically acceptable material that is included in a drug composition to enhance the pharmaceutical (including manufacturing and shelf-stability) and/or pharmacological properties thereof. Pharmaceutical excipients include, but are not limited to, adjuvants, surfactants, stabilizers, morphology modifiers, porogens, diluents, carriers, solubilizers, antioxidants, lubricants (or glidants), binders, disintigrants, and mixtures thereof.

### B. Drugs.

Any of a variety of drugs or pharmaceutical compounds can be used to carry out the present invention, including but not limited to antidiabetics, analgesics, antiinflammatory agents, antirheumatics, antihypotensive agents, antihypertensive agents, psychoactive drugs, tranquillizers, antiemetics, muscle relaxants, glucocorticoids, agents for treating ulcerative colitis or Crohn's disease, ant/allergies, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals, chemotherapeutic agents and amino acids. Examples of suitable active ingredients are acarbose, antigens, beta-receptor blockers, non-steroidal antiinflammatory drugs {NSAIDs], cardiac glycosides, acetylsalicylic acid, virustatics, aclarubicin, acyclovir, cisplatin, actinomycin, alpha- and beta-sympatomimetics, dmeprazole, allopurinol, alprostadil, prostaglandins, amantadine, ambroxol, amlodipine, methotrexate, aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytabarine, dapiprazole, desogestrel, desonide, dihydralazine, diltiazem, ergot alkaloids, dimenhydrinate, dimethyl sulphoxide, dimeticone, domperidone and domperidan derivatives, dopamine, doxazosin, doxorubizin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenytoin, riluzoles, risedronate, sildenafil, topiramate, macrolide antibiotics, oestrogen and oestrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflurnic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentoxifylline, perphenazine, pethidine, plant extracts, phenazone, pheniramine, barbituric acid derivatives, phenylbutazone, phenytoin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tetracyclins, teryzoline, theobromine, theophylline, butizine, thiamazole, phenothiazines, thiotepa, tiagabine, tiapride, propionic acid derivatives, ticlopidine, timolol, tinidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobuterol, tyramine, tyrothricin, urapidil, ursodeoxychoiic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, zotipinc and the like. For the purposes of the current invention, drugs may also include food products such as neutraceuticals, flavenoids, and the like. See, e.g., U.S. Pat. No. 6,897,205; see also U.S. Pat. No. 6,838,528; U.S. Pat. No. 6,497,729.

The active ingredients may, if desired, also be used in the form of their pharmaceutically acceptable salts or derivatives, and in the case of chiral active ingredients it is possible to employ both optically active isomers and racemates or mixtures of diastereoisomers. If desired, the compositions of the invention may also comprise two or more active pharmaceutical ingredients.

The drug or active ingredient may be in any physical form, such as crystalline (including sernicrystalline) and amorphous.

### C. Polymers.

Any suitable polymer can be used to carry out the present invention, including but not limited to: natural and synthetic polymers, gelatin, chitosan, dextrin, cyclodextrin, Poly(urethanes), Poly(siloxanes) or silicones, Poly(acrylates) such as poiy(methyl methacrylate), poly(butyl methacrylate), and Poly(2-hydroxy ethyl methacrylate), Poly(vinyl alcohol) Poly(olefinds) such as poly(ethylene), poly(isoprene), halogenated polymers such as Poly(tetrafluoroethylene)—and derivatives and copolymers such as those commonly sold as Teflon® products, Poly(vinylidine fluoride), Poly(vinyl acetate), Poly(vinyl pyrrolidone), Poly(acrylic acid), Polyacrylamide, Poly(ethylene-co-vinyl acetate), Poly(ethylene glycol), Poly(propylene glycol), Poly(methacrylic acid); etc.

Suitable polymers also include absorbable and/or resorbable polymers including the following, combinations, copolymers and derivatives of the following: Polylactides (PLA), Polyglycolides (PGA), Poly(lactide-co-glycolides) (PLEA), Polyanhydrides, Polyorthoesters, PoIy(N-(2-hydroxypropyl) methacrylamide), Poly(1-aspartamide), etc.

### D. Solvents.

Solvents that may be used to carry out the present invention are, in some embodiments, gases (that is, compounds that are in the form of a gas at atmospheric pressure and 25° C.). Examples of such solvents include but are not limited to carbon dioxide, ammonia, water, methanol, ethanol, ethane, propane, butane, pentane, dimethyl ether, xenon, sulfur hexafluoride, halogenated and partially halogenated materials such as chlorofluorocarbons, hydrochlorofluorocarbons, hydro fluorocarbons, perfluorocarbons (such as perfluoromethane and perfuoropropane, chloroform, trichloro-fluoromethane, dichloro-difluoromethane, dichloro-tetrafluoroethane) and mixtures thereof. Carbon dioxide is preferred.

The solvent may be utilized per se or a cosolvent may be included therewith (e.g., in an amount of from 0.01 or 0.1 to 20 or 30 percent by weight or more). Examples of cosolvents include, but are not limited to, water and organic co-solvents. The organic co-solvent may be one compound or a mixture of two or more ingredients. The organic co-solvent may be or comprise an alcohol (including dials, triols, etc.), ether, amine, ketone, carbonate, or alkanes, or hydrocarbon (aliphatic or aromatic) The organic co-solvent may be a mixture of compounds, such as mixtures of alkanes as given above, or mixtures of one or more alkanes in combination with additional compounds such as one or more alcohols as described above. (e.g., from 0 or 0.1 to 5% of a C1 to C15 alcohol (including dials, triols, etc.)). See, e.g., .U.S Pat. No. 6,669,785. The solvent may optionally contain a surfactant, as also described in (for example) U.S. Pat. No. 6,669,785.

The solvent is preferably provided in compressed form as a liquid (including near-supercritical fluids) or as a supercritical fluid, these two forms together sometimes being referred to as a “densified” fluid or “densified” gas. See, e.g., U.S. Pat. Nos. 6,860,123; 6,837,611; and 6,755,871.

### E. Excipients.

Numerous pharmaceutical excipients that may be used to carry out the present invention are known. See, e.g., U.S. Pat. Nos. 6,767,558; 6,720,003; 6,710,059; and 6,649,627. Comprehensive examples are included in the Handbook of Pharmaceutical Excipients, edited by Raymond. Rowe, Paul Sheskey and Paul Weller (4^{th} Ed. 2003). Among other things, the drug-polymer composition may contain pharmaceutical excipients materials for: 1) enhancing the stability of the drug, 2) modifying the ultimate morphology of the drug or polymer, or drug polymer composite 3) inserting a porogen into the composite for subsequent removal in or during dense fluid processing, 4) improving the solubility characteristics of the drug in-vitro and in-vivo. Ideally these excipients are classified as Generally Regarded As Safe (GRAS) materials by the US Food and Drug Administration (FDA).

In category ‘2’ above the excipient serves to stabilize the drug material. A primary example is represented by the use of sugars and other carbohydrates to stabilize proteins and peptides in pharmaceutical formulations. In the current invention one particularly useful sugar derivative is Sucrose OctaAcetate (SOA) which can serve to stabilize proteins in solution or in the solid state during compounding of the drug with the polymer. The SOA may also serve to benefit the composite in downstream processing described below.

In category ‘2’ above the excipient serves to modify the morphology of the drug or polymer or the composite during and after processing with a dense gas fluid. One highlighted advantage to using dense gas fluids for processing drug-polymer composites relates to the “plasticizing” effect of the fluid (such as supercritical carbon dioxide) on the polymer. The fluid essentially permeates the free-volume of the polymer micro-structure lowering the glass transition temperature of the amorphous polymer and enhancing particle fusion at temperature much lower than those needed for heat bonding or fusion. This enhanced flow allows for suitable cohesion or adhesion of the formulated drug-polymer composite creating a semi-rigid composite product. The inclusion of excipients such as SOA may also serve to further plasticize the polymer thus enhancing the particle fusion and the overall solid-state integrity of the final composite.

In category ‘3’ above the excipient serves as a removable material (porogen) during the dense fluid processing step. For non-absorbable polymers it may be desirable to create increased surface area to affect drug removal in-vivo. By inclusion of the excipient material during the compounding step, a porous or semi-porous structure is created upon exposure to the dense fluid. In this case, the excipient is extracted from the formed composite leaving a micro- or nano-porous internal structure after completed dense fluid processing. One particular excipient of interest is SOA. Sucrose octaacetate is know to be soluble in dense carbon dioxide and in this case may serve as a stabilizer, a plasticizer, and a porogen. Other partially or fully acetylated sugars and carbohydrates may also be employed for these same purposes.

In category ‘4’ above the excipient increases the solubility of the drug as measure in-vitro and as applied in-vivo by preventing drug aggregation/agglomeration and by increasing the hydration capacity of the drug particle in-situ. Many drugs have poor aqueous solubility and therefore limited efficacy based on there ability to reach sufficient levels in the blood. Aside from particle size control (smaller particle size equals better dissolution profiles) excipients are used to prevent particle agglomeration and to enhance dissolution characteristics by increasing hydration in and around the particle. Noteworthy examples useful in the current invention include dextrin and its derivatives, other carbohydrates and simple sugars, and partially or fully acetylated sugars such as SOA.

As outlined above the excipient may serve one or several of the purposes described.

Other useful excipients include surfactants. Ideally, these surfactants are classified as GRAS materials by the FDA. Suitable examples include but are not limited to sorbitan monooleate, Twean® trademarked surfactants, soy derived surfactants, and fatty acid derived GRAS surfactants. These surfactants may serve one or multiple roles as described above in this section.

As indicated above, SOA and other such hydrophobically derivatized carbohydrates (HDCs) can be utilized as the pharmaceutical excipient. HDCs are a wide variety of hydrophobically derivatized carbohydrates where at least one hydroxyl group is substituted with a hydrophobic moiety including, but not limited to, esters and ethers. Numerous examples of suitable HDCs and their syntheses are described in Developments in Food Carbohydrate, C. K. Lee, Applied Science Publishers, London (2d Ed. 1980) and PCT publication No. 96/03978. Other syntheses are described in, for example, Akoh et al. (1987) J. Food Sci. 52:1570;Khan et al. (1933) Tetra. Letts 34:7767; Khan (1984) Pure & Appl. Chem. 56:833-844; and Khan et al. (1990) Carb. Res. 198:275-283. Specific examples of HDCs include, but are not limited to, sorbitol hexaacetate (SHAC), alpha-glucose pentaacetate (alpha-GPAC), beta-glucose pentaacetate (beta-GPAC), 1-O-Octyl-.beta.-D-glucose tetraacetate (OCTA), trehalose octaacetate (TOAC), trehalose octapropionate (TOP), trehalose octa-3,3,dimethylbutyrate (TO33DMB), trehalose diisobutyrate hexaacetate, trehalose octaisobutyrate, lactose octaacetate, sucrose octaacetate (SOAC), cellobiose octaacetate (COAC), raffinose undecaacetate (RUDA), sucrose octapropanoate, cellobiose octapropanoate, raffinose undecapropanoate, tetra-O-methyl trehalose, trehalose octapivalate, trehalose hexaacetate dipivalate and di-O-methyl-hexa-O-actyl sucrose and mixtures thereof. See, e.g., U.S. Pat. No. 6,517,860.

### F. Methods of Making and Using.

The method of the invention may be carried out by first, combining the drug with the polymer and optionally an excipients) to form a mixture. This mixing step may be carried out by any suitable technique or in any suitable apparatus, such as in a blender, extruder, etc. Typically both the drug and the polymer are provided in solid particulate form, and hence the mixture so formed will also be in the form of a solid.

Typically the polymer and the drug particles range between 0.02 and 50 microns in size. In some embodiments the particle size is in a larger size range than the drug. In this case the polymer may range from 0.2 micron and 50 microns and the drug from 0.02 to 20 microns.

Next, the mixture is contacted under pressure with a compressed gas solvent as described above to form the composite material. Without wishing to be bound to any particular theory of the invention, it is believed that the compressed gas solvent is at a pressure sufficient to reduce the viscosity of the polymer material, trapping the fluid insoluble drug material in the polymer matrix as polymer particles fuse with adjacent polymer particles and hence form the drug/polymer composite article. As contrasted with other art utilizing dense fluid gases such as carbon dioxide at high pressures, many drugs, particularly protein-based drugs, are not soluble in the dense fluid and therefore arc not efficiently infused into polymer matrices. In the current invention the drug, such as a protein-based therapeutic may remain largely unchanged as the polymer particle fuse around the drug particles. Depending upon the specific manner in which this step is carried out the drug/polymer composite can be in the form of discrete particles (which may for example he the same size but likely larger than the polymer particles previously provided) or may be in the form of a shaped article. Ideally, the composite mixture is used in conjunction with a mechanical article such as a mold or a template and the final composite article takes on the shape or general shape of that mold or template. So in working practice the mixture of the drug, polymer and excipients is added to a three-dimensional article, mechanically constrained such that the particles of both the drug and the polymer axe immobilized. The supercritical fluid at the desired pressure and temperature is then allowed to permeate the three-dimensional article such to effect the fusion of the polymer particles without extraction or removal of either the drug or the polymer from the mechanical article. Finally the fluid is removed from the mechanical article by reducing the pressure to ambient levels and the final composite is then removed from the template as a semi-rigid solid composite. In general, this contacting step is carried out at a pressure between 500 and 15,000 psig and a temperature of between 20 C and 175° C. Most preferably the contacting step is carried out at between 1100 and 5000 psig at a temperature between 30 C and 110° C.

The step of combining the mixture with the solvent can be carried out by any suitable technique or in any suitable apparatus, such as in an extruder (which may be the same or different from the extruder noted above), mold (e.g., injection mold, blow mold, compression mold, etc.), reaction vessel, etc. A shaped article as described herein may, in some embodiments, be formed concurrently with this combining step, for example when the combining is carried out in a mold, or when the combining is carried out in an extruder and the composite formed therein then extruded through a die. In other embodiments, however, the shaped article will be formed in a subsequent step. Such subsequent forming may likewise be carried out by any suitable technique such as by spraying or dipping a pre-formed substrate with the composite material (e.g., to form a stent or biomedical implant). By use of a subsequent extruder or mold, etc.

The drug/composite material may comprise, consist of, or consist essentially of:

from 0.01 or 0.1 percent to 40, 50, or 60 percent by weight of drug (which may be a single compound or a combination of different active agents); and

from 40 or 50 percent to 99.9 or 99.99 percent by weight of polymer;

optionally, from 0.01 or 0.1 percent to 20 or 30 percent pharmaceutical excipient.

In some embodiments, the physical form of the drug in the composite is substantially the same as the physical form of the drug before the combining step (b). For example, a drug initially provided in crystalline form remains in crystalline form in the composite; a drug initially provided in amorphous form remains in amorphous form in the composite; etc.

In some embodiments, the composite is porous (this term including “semiporous”), with porous composites being made by inclusion of a porogen as a pharmaceutical excipient and subsequent removal of at least a portion thereof by an appropriate solvent (e.g., organic solvents; densified carbon dioxide solvent compositions as described herein) thereof after formation of the composite, in accordance with known techniques. In some embodiments the porogen is an SOA or other such hydrophobically derivatized carbohydrate as described above.

Secondary coatings. Drug/polymer composites prepared as described above may optionally be coated (e.g., by spraying, dipping, or any suitable technique) with a second material to aid in the subsequent binding, forming, dispersion, structure or drug-elution profile of the drug/polymer composite. This second material can be any of several different chemical functionalities and several different functions in the resulting drug/polymer composite material. For example, the second material can be a pharmaceutical excipient, providing a means to alter the pharmacological effect of the drug or providing a means to alter the release profile of the drug-delivery. In some embodiments the second material can be a CO₂-philic material. In this case an additional process step can be utilized where after compressive forming of the part, a second condition of compressed fluid can be used to remove the CO₂-philic material, thereby forming pores in and rendering porosity to the formed part.

The present invention is explained in greater detail in the following non-limiting Examples.

### EXAMPLE 1

### Preparation of a Drug Polymer Composite

### Article Uusing Supercritical Fluid Processing

A cylindrical composite article consisting of 3 parts poly(butyl methacrylate), 2 parts recombinant Human Growth hormone (rHGh), and 1 part sucrose octaacetate is created in the following manner. Spherical emulsion prepared poly(butyl methacrylate) of an average size range of 3.0 microns is blended with lyophilized HGh with an average particle size of 1.0 microns using an ultrasonic mixer. Dry sucrose octaacetate powder in the appropriate ratio is then added under constant mixing. The resulting formulation is then added to a cylindrical hollow mold constructed from sintered metal creating a fluid permeable three-dimensional article with an average pore size of 0.2 microns. The cylinder is open on both ends. Prior to the addition of the drug-polymer composition to the mold, one end is closed off using a matching cap designed to lock in place at the end of the cylinder. Once added to the mold, the composition is then mechanically compressed using a metal plunger matching the approximate inner diameter of the cylinder minus 0.001-inch to remove the majority of the free-volume. The other end of the cylinder is then closed using an end cap that locks in place constraining the composition in three dimensions. The mold containing the polymer drug composition is then placed in a sterile pressure vessel to which 99.99% pure carbon dioxide is added to a pressure of 4000 psig at a temperature of 80 C. The article is maintained in the CO2 environment at this temperature for 20 minutes after which the vessel is vented to atmospheric conditions. The mold is then removed from the vessel and the end caps are removed. The drug-polymer composite is then removed from the mold using a metal plunger fed from the open top of the mold thus pushing the composite out the bottom as the cylinder is mechanically restrained. Upon inspection the sample is a semi-rigid solid article in the shape of the mold. Upon thorough analysis of the polymer drug composite using Scanning Electron Microscopy (SEM) and routine chemical analysis it is determined that the solid article consists of a porous network of fused polymer particles with protein residing largely between adjacent fused particles and in void spaces created by the partial extraction of the sucrose octaacetate. Upon detailed morphological and chemical examination of the composite it is determined that the porous structure is largely inter-connected and partially opened to the outer surface of the article and the ratio of polymer to drug to sucrose octaacetate was 3:2:0.2 indicating substantial removal of the sucrose derivative during fluid processing.

### EXAMPLE 2

### Preparation of a Drug Polymer Composite

### Article Using Supercritical Fluid Processing

A cylindrical composite article consisting of 4 parts poly(butyl methacrylate), 2 parts recombinant Human Growth hormone (rHGh), and 2 part sucrose octaacetate is created in the following manner. Spherical emulsion prepared poly(butyl methacrylate) of an average size range of 10.0 microns is blended with lyophilized HGh with an average particle size of 1.0 microns using an ultrasonic mixer. Dry sucrose octaacetate powder in the appropriate ratio is then added under constant mixing. The resulting formulation is then added to a cylindrical hollow mold constructed from sintered metal creating a fluid permeable three-dimensional article with an average pore size of 0.2 microns. The cylinder is open on both ends. Prior to the addition of the drug-polymer composition to the mold, one end is closed off using a matching cap designed to lock in place at the end of the cylinder. The mold containing the polymer-drug-excipient mixture is then added to a pressure vessel equipped with a mechanical device designed with a piston actuator to exert pressure on the open end of the mold. The sealed pressure vessel is then filled with supercritical CO2 to a pressure of 3000 psi at a temperature of 80 C. After 5 minutes at static pressure and temperature, the piston is actuated to apply mechanical pressure through the open end of the mold compressing the composition with 25 lbs-(in²)⁻¹ of mechanical force. After 5 minutes of mechanical compression and exposure to CO2 at a pressure of 3000 psi (80C) the CO2 is vented from the chamber and the piston is removed from the open end of the cylindrical mold. The drug-polymer composite is then removed from the mold using a metal plunger fed from the open top of the mold thus pushing the composite out the bottom as the cylinder is mechanically restrained. Upon inspection the sample is a semi-rigid solid article in the shape of the mold.

The foregoing is illustrative of the present invention, and is not to be construed as limiting thereof. The invention is defined by the following claims, with equivalents of the claims to be included therein.

1. A method of forming a biomedical implant comprising a drug/polymer composite material, the method comprising the steps of:
(a) combining a solid particulate macrolide immunosuppressive drug material in crystalline form having a therapeutic effect with a solid particulate polymer material, and optionally with a pharmaceutical excipient, to intersperse drug particles within polymer particles;
(b) constraining the interspersed drug and polymer in a mold so that the drug particles and the polymer particles are immobilized; and then
(c) permeating the mold with a compressed gas solvent to contact the interspersed drug and polymer particles at a pressure sufficient to reduce the viscosity of said polymer material such that said polymer particles are fused to one another around the drug particles to capture said drug particles therebetween without mobilization of the drug particles and to form a drug/polymer composite material from said particulate mixture, the compressed gas solvent being a densified gas or a near supercritical or supercritical fluid, wherein the biomedical implant comprising the polymer-drug composite is formed without physically or chemically changing the state of the drug during processing.

2. The method of claim 1, wherein said biomedical implant is a drug depot.

3. The method of claim 1, wherein said drug is a protein or peptide.

4. The method of claim 1, wherein said composite material comprises:
from 0.01 percent to 50 percent by weight of drug;
from 50 to 99.99 percent by weight of polymer; and
optionally, from 0.01 to 30 percent by weight of pharmaceutical excipient.

5. The method of claim 1, wherein said pharmaceutical excipient is present.

6. The method of claim 5, wherein said pharmaceutical excipient is selected from the group consisting of adjuvants, surfactants, stabilizers, morphology modifiers, porogens, diluents, carriers, solubilizers, antioxidants, lubricants, binders, disintigrants, and mixtures thereof.

7. The method of claim 5, wherein said pharmaceutical excipient is a hydrophobically derivatized carbohydrate.

8. The method of claim 7, wherein said hydrophobically derivatized carbohydrate is selected from the group consisting of sorbitol hexaacetate, alpha-glucose pentaacetate, beta-glucose pentaacetate, 1-0-Octyl-beta-D-glucose tetraacetate, trehalose octaacetate, tetralose octapropionate, trehalose octa-3,3,dimethylbutyrate, trehalose diisobutyrate hexaacetate, trehalose octaisobutyrate, lactose octaacetate, sucrose octaacetate, cellobiose octaacetate, raffinoso undecaacetate, sucrose octapropanoate, cellobiose octapropanoate, raffinose undecapropanoate, tetra-0-methyl trehalose, trehalose octapivalate, trehalose hexaacetate dipivalate and di-0-methylhexa-0-actyl sucrose and mixtures thereof.

9. The method of claim 1, further comprising the step of coating said composite material with a secondary material.

10. The method of claim 1, wherein said excipient is a porogen, said method further comprising the step of contacting said composite material to a solvent to at least partially solubilize said porogen and form pores in said composite material.

11. A drug/polymer composite material produced by the process of claim 1.

12. The composite of claim 11, wherein said composite is porous.

13. A method of treating a subject with a drug, comprising administering a drug/polymer composite material of claim 11 to said subject in an amount effective to treat said subject with said drug.

14. A shaped article comprising a drug/polymer composite material of claim 11.

15. The shaped article of claim 14, wherein said shaped article is a stent, drug depot, or biomedical implant.

16. The shaped article of claim 14, wherein said shaped article is a porous subcutaneous drug depot.

17. The method of claim 1 wherein step (c) comprises contacting the drug and polymer with supercritical carbon dioxide.
